(19) United States
(12) Patent Application Publication
(10) Pub. No.: US 2023/0255864 A1
(43) Pub. Date: Aug. 17, 2023

(54) NOVEL COMPOSITIONS COMBINING HEMP DERIVATIVES AND AT LEAST ONE PEPTIDE AND USE THEREOF

(71) Applicant: FRANCE COSMEPHYL-LAB, Paris (FR)

(72) Inventors: Michel HOCQUAUX, Paris (FR); Jean-Christophe AUBAGNAC, Saint Jean (FR)

(21) Appl. No.: 18/005,123

(22) PCT Filed: Jul. 13, 2021
(86) PCT No.: PCT/EP2021/069472

§ 371 Date: Jan. 11, 2023

(30) Foreign Application Priority Data
Jul. 15, 2020 (FR) 2007406

(51) Int. Cl. A61K 8/34; A61K 8/60; A61K 8/29; A61K 8/9789; A61Q 17/04

(52) U.S. Cl. CPC A61K 8/347; A61K 8/60; A61K 8/29; A61K 8/9789; A61Q 17/04

(57) ABSTRACT

The present invention relates to an association comprising or consisting of a mixture of cannabidiol or cannahigerol and palinitoyl tripeptide-8, and to the use thereof for treating or soothing sensitive and/or reactive skins or as an after-sun ointment for soothing solar erythema.

### FIELD OF THE INVENTION

The present invention relates to new compositions combining hemp derivatives and at least one peptide and their use in cosmetics, as well as their pharmaceutical use , in particular in dermatology, in particular for the treatment of irritation of sensitive skins and actinide erythema or “sunburns”.

### STATE OF THE PRIOR ART

The skin is a vital organ in its own right which can become very fragile when it is subjected to external aggressions such as environmental stresses. There are very sensitive skins prone to inflammation in a more or less chronic way and without necessarily having been subjected to external aggressions. The symptoms of sensitive skins are more or less diffuse and localized redness, itching, tightness and sometimes localized swelling. These feelings of discomfort can be due to external aggressions such as UV rays or to internal factors. In all cases, they manifest themselves in the form of micro-inflammation or revealed inflammation.

Skin inflammation caused by external aggressions is the cause of skin sensitization. It is a complex phenomenon. Indeed, neurogenic skin inflammation is defined as the induction and/or amplification of a primary inflammatory process by the nerve endings, thus it is an inflammation of the skin induced by the activation of intra-epidermal nerve fibers which secrete neuropeptides such as substance P. Neurogenic cutaneous inflammation is involved in sensitive skins, reactive intolerant skins and even pruriginous inflammatory dermatoses. Pruritus is defined as a sensation that causes the need to scratch. Research has revealed specific receptors; these pruriceptors secrete neuropeptides: substance P, the “calcitonin gene related peptide” called CGRP and the “vasoactive intestinal peptide” called VIP. The role of proteases in the induction of pruritus has also been established; their PAR-2 receptor has been defined as the second pathway of pruritus activation (Misery et al. Nat.Rev. Neurol 10, 408-416, 2014). Thus, when the tolerance threshold is exceeded, the activated keratinocytes locally initiate the inflammatory response via the initial release of pro-inflammatory mediators such as interleukins 1α (IL 1-α) and 6 (IL 6), neurokinins such as the growth factor NGF (“Nerve Growth Factor”), and TNFα. The release of IL1α induces an immediate cellular reaction, activation of enzymes forming lipid mediators of inflammation, leukotrienes and prostaglandins. In addition to this inflammatory reaction, there is a neurogenic mechanism of skin sensitivity involving the sensory nerves of the epidermis. The release of NGF by the keratinocytes will cause an overexpression of the neuronal receptor TRPV1 (“Transient Receptor Potential Vanilloid 1”) which is a pain receptor.

One of the main factors of aggression of the skin is considered to be the UVA and UVB ultraviolet rays of the sun. UVB rays reaching the surface of the skin can cause, along with tanning, burns and signs of aging. UVA rays can penetrate deeper into the skin, causing the release of free radicals, causing DNA changes. Thus UV rays have a pro-oxidant effect. UV exposure causes significant damage to the skin and has deleterious short and long terms consequences. In the short term, exposure of the skin to significant doses of UV is responsible for “sunburn”.

“Sunburn” or erythema induced by solar radiation, or actinic erythema, clinically corresponds to histological characteristics marked by the presence of dyskeratotic cells or “sunburn cells”, corresponding to keratinocytes in apoptosis. Actinic erythema is mainly linked to UVB but also to the action of UVA.

The consequences of UV-induced erythema are multiple, involving DNA damage, generation of reactive oxygen species (ROS) and a set of inflammatory mediators.

Thus, to effectively fight against “sunburn”, a product must present an action against inflammation mediators, activated oxygen species and repair DNA damage.

Thus, to be effective against inflammation of the skin, sensitive skins or erythema induced by solar irradiation, a skin treatment must act on several factors, including the limitation of the amplification of the inflammation, and the action on neurogenic inflammation.

It is known that skin cells have neurotransmitter receptors allowing them to modulate all the properties of the skin such as cell growth, production of inflammatory mediators, immunity or vasodilation.

The nervous system of the skin has in particular numerous cannabinoid receptors.

Among more than sixty known plant cannabinoids, cannabidiol (CBD) or 2-R1R,6R)-6-isopropenyl-3-methylcyclohex-2-en-1-yl1-5-pentylbenzene-1,3-diol, is the second most abundant cannabinoid in hemp. Cannabidiol can be extracted from several varieties of hemp, including Cannabis sativa, indica, and ruderalis. In particular, it can be extracted pure from genetically modified hemp plants or synthesized in the laboratory.

Its structure is shown below.

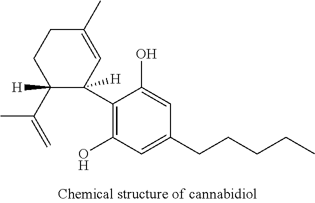 [SMILES: C=C(C)[C@@H]1CCC(C)=C[C@H]1c1c(O)cc(CCCCC)cc1O]

It is a lipophilic substance, it does not exhibit a psychoactive effect.

Cannabidiol CBD shows potential in reducing the inflammatory response of the skin. Indeed, studies have shown that it has anti-inflammatory (Burstein S., Bioorganic & Medicinal Chemistry, Vol 23, 2015, p 1377-1385) and antioxidant properties (Booz G. W.; Free Radical Biology and Medicine, Vol 51), September 2011, p 1054-1061). Cannabigerol (CBG) is another cannabinoid, present in low content, around 1%, in hemp. Its structure is shown below.

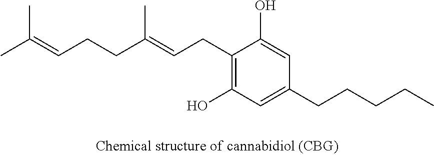 [SMILES: CCCCCc1cc(O)c(C/C=C(\C)CCC=C(C)C)c(O)c1]

Thus, cannabidiol and cannabigerol help relieve skin conditions such as inflammation, itching, pruritus. Other substances such as biomimetic peptides can have an effect on the nervous system of the skin. Indeed, biomimetic peptides mimic the action of natural peptides, while being biocompatible.

Among the biomimetic peptides derived from a neurotransmitter with anti-inflammatory properties, one can cite palmitoyl tripeptide-8.

The “palmitoyl tripeptide-8” or (L-Argininamide, N-(1-oxohexadecyl)-L-histidyl-D-phenylalanyl) is a tripeptide of sequence “Palm-His-(D)Phe-Arg-NH₂” in which “Palm” represents the radical corresponding to palmitic acid. The structure of the molecule, whose CAS number is [936544-53-5] is shown below. It can be obtained according to the method described in application FR 2 870 243.

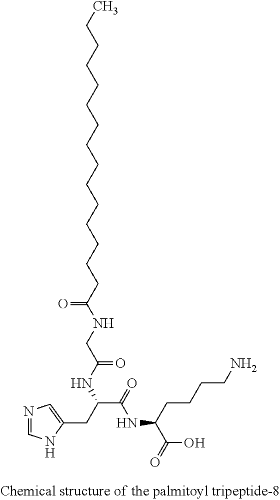 [SMILES: CCCCCCCCCCCCCCCC(=O)NCC(=O)N[C@@H](Cc1cnc[nH]1)C(=O)N[C@@H](CCCCN)C(=O)O]

### Technical Problem

There is a need for compositions making it possible to treat and soothe inflammatory reactions of the skin.

One of the aims of the invention is to propose compositions making it possible to treat and soothe the inflammations of sensitive skins, the inflammatory component of which is revealed.

Another aim of the invention is to provide compositions for treating and soothing erythema induced by solar irradiation or “sunburn”.

Another aim of the invention is to provide compositions, in particular topical, for soothing and treating inflammation based on natural products, including for example plant extracts, and/or based on biomimetic products, inspired by nature.

One of the aims of the invention is to propose topical compositions capable of being used as a soothing after-sun on healthy skins.

### INVENTION

Surprisingly, the Inventors have discovered that the association of cannabidiol or cannabigerol, and palmitoyl-tripeptide-8 turns out to present a synergy between the substances resulting in remarkable soothing properties.

Indeed, the association of cannabidiol and palmitoyl-tripeptide-8 exhibits a synergistic anti-inflammatory activity in conjunction with a reduction in cytotoxicity.

A first object of the present invention is an association comprising or consisting of a mixture of cannabidiol or cannabigerol and palmitoyl tripeptide 8.

The association according to the present invention is a mixture of a natural substance and a biomimetic peptide. More specifically, the present invention is a mixture of a particular cannabinoid, cannabidiol or cannabigerol, and a palmitoyl tripeptide with particular N-terminal end, palmitoyl tripeptide 8.

According to another particular embodiment, the cannabidiol or cannabigerol used is of natural origin and comes in particular from plant extracts of different varieties of hemp, in particular Cannabis sativa, indica or ruderalis.

According to another particular embodiment, the cannabidiol or cannabigerol used is isolated by methods known to those skilled in the art, for example comprising the collection of plant materials, extraction and purification.

According to another particular embodiment, cannabidiol or cannabigerol is synthesized by methods known to those skilled in the art. According to another particular embodiment, the cannabidiol or cannabigerol used comes from hemp extracts containing less than 0.2% THC.

According to another particular embodiment, the cannabidiol or cannabigerol used comes from plants or extracts in accordance with the national laws in force.

According to another particular embodiment, the present invention relates to an association comprising or consisting of a mixture of cannabidiol or cannabigerol and palmitoyl tripeptide-8, in particular of a mixture of:
cannabidiol or cannabigerol,
palmitoyl tripeptide-8,
butylene glycol,
dextran,
and water.

According to another particular embodiment, the present invention relates to an association comprising or consisting of a mixture of cannabidiol and palmitoyl tripeptide-8, in particular of a mixture of:
cannabidiol,
palmitoyl tripeptide-8,
butylene glycol,
dextran,
and water.

According to another particular embodiment, the present invention relates to an association comprising or consisting of a mixture of cannabigerol and palmitoyl tripeptide-8, in particular of a mixture of:
cannabigerol,
palmitoyl tripeptide-8,
butylene glycol,
dextran,
and water.

According to another particular embodiment, the present invention relates to an association comprising or consisting of a mixture of cannabidiol, cannabigerol and palmitoyl tripeptide-8, in particular a mixture of:
cannabidiol,
cannabigerol,
palmitoyl tripeptide-8,
butylene glycol,
dextran,
and water.

Butylene glycol or butane-1,3-diol is known to be used in cosmetics as a humectant in skin care. It prevents the product from drying out and makes the formulation more resistant to humidity.

Dextran is a branched polymer of dextrose. In the cosmetic field, the latter has the function of being a fixing agent and a thickener.

Palmitoyl Tripeptide-8 is commercially available in a form that can be used in the context of the present invention. Palmitoyl Tripeptide-8 is in particular marketed under the trade name of Neutrazen™ by the company Lucas Meyer. Neutrazen™ (INCI name: Water (and) Butylene Glycol (and) Dextran (and) Palmitoyl Tripeptide-8) is a solution of palmitoyl tripeptide-8 in a mixture of butylene glycol, dextran and water.

The use of the said commercial product Neutrazen™ therefore leads to an association comprising dextran and butylene glycol.

According to a particular embodiment, the subject of the present invention is an association as defined above, in which:
the cannabidiol or cannabigerol is present in an amount of 0.01% to 5%, preferably 0.25% to 1% by weight percent
the palmitoyl tripeptide-8 is present in an amount of 2 ppm to 20 ppm, preferably 4 ppm to 10 ppm by weight.

According to a particular embodiment, the subject of the present invention is an association as defined above, in which:
the cannabidiol is present in an amount of 0.01% to 5%, preferably of 0.25% to 1% by weight percent
the palmitoyl tripeptide-8 is present in an amount of 2 ppm to 20 ppm, preferably 4 ppm to 10 ppm by weight.

According to a particular embodiment, the subject of the present invention is an association as defined above, in which:
the cannabigerol is present in an amount of 0.01% to 5%, preferably of 0.25% to 1% by weight percent
the palmitoyl tripeptide-8 is present in an amount of 2 ppm to 20 ppm, preferably 4 ppm to 10 ppm by weight.

Below 0.01%, the effect of cannabidiol or cannabigerol is insufficient. Beyond 5%, the price of the raw material does not allow the marketing of a profitable product.

According to another particular embodiment, the present invention relates to an association as defined above, in which the cannabidiol or cannabigerol is in an amount of 0.01% to 5%, preferably from 0.25% to 1% in percentage by weight relative to the total weight.

According to another particular embodiment, the present invention relates to an association as defined above, in which the cannabidiol is in an amount of 0.01% to 5%, preferably of 0.25% to 1% in percentage by weight relative to total weight.

According to another particular embodiment, the present invention relates to an association as defined above, in which the cannabigerol is in an amount of 0.01% to 5%, preferably of 0.25% to 1% in percentage by weight relative to total weight.

The term “from 0.01 to 5%” also means the following ranges: from 0.01 to 0.025%; from 0.025 to 0.05%; from 0.05 to 0.1%; from 0.1 to 0.5%; from 0.5 to 1.0%; from 1.0 to 1.5%; from 1.5 to 2.0%; from 2.0 to 2.5%; from 2.5 to 3.0%; from 3.0 to 3.5%; from 3.5 to 4.0%; from 4.0 to 4.5%; from 4.5 to 5.0%; and in particular about 0.5%.

The term “from 0.25 to 1%” also means the following ranges: from 0.25 to 0.50%; from 0.5 to 0.75%; from 0.75 to 1.0%; and in particular about 0.50%.

According to another particular embodiment, the present invention relates to an association as defined above, in which the palmitoyl tripeptide-8 is present in an amount of 2 ppm to 20 ppm, preferably of 4 ppm to 10 ppm by weight.

The term “from 2 ppm to 20 ppm” of palmitoyl tripeptide-8 also means the following ranges: from 2 ppm to 5 ppm; from 5 ppm to 10 ppm; from 10 ppm to 15 ppm; from 15 ppm to 20 ppm; and in particular about 10 ppm.

The term “from 4 ppm to 10 ppm” of palmitoyl tripeptide-8 also means the following ranges: from 4 ppm to 6 ppm; from 6 ppm to 8 ppm; from 8 ppm to 10 ppm.

According to a particular embodiment, the subject of the present invention is an association as defined above, comprising:
from 10 mg to 5.0 g of cannabidiol or cannabigerol, preferably from 250 mg to 1 g,
from 0.01% to 5.0% of a Neutrazen™ solution, preferably from 1.0% to 2.5%.

According to a particular embodiment, the subject of the present invention is an association as defined above, comprising:
from 10 mg to 5.0 g of cannabidiol, preferably from 250 mg to 1 g,
from 0.01% to 5.0% of a Neutrazen™ solution, preferably from 1.0% to 2.5%.

According to a particular embodiment, the subject of the present invention is an association as defined above, comprising:
from 10 mg to 5.0 g of cannabigerol, preferably from 250 mg to 1 g,
from 0.01% to 5.0% of a Neutrazen™ solution, preferably from 1.0% to 2.5%.

The term “from 10 mg to 5 g” means the following ranges: from 10 to 25 mg; from 25 to 50 mg; from 50 to 100 mg; from 100 to 500 mg; from 500 mg to 1.0 g; from 1.0 to 1.5 g; from 1.5 to 2.0 g; from 2.0 to 2.5 g; from 2.5 to 3.0 g; from 3.0 to 3.5 g; from 3.5 to 4.0 g; from 4.0 to 4.5 g; from 4.5 to 5.0 g; in particular about 1.0 g.

The term “from 250 mg to 1 g” also means the following ranges: from 250 to 400 mg; from 400 to 600 mg; from 600 to 800 mg; from 800 to 1.0 g; in particular about 500 mg.

The Neutrazen™ product from Lucas Meyer Cosmetics is advantageously used in an amount comprised from 0.01% to 5.0%, as a percentage by weight relative to the total weight, or from 0.01 to 0.025%; from 0.025 to 0.05%; from 0.05 to 0.1%; from 0.1 to 0.25%; from 0.25 to 0.5%; from 0.5 to 1.0%; from 1.0 to 1.5%; from 1.5 to 2.0%; from 2.0 to 2.5%; from 2.5 to 3.0%; from 3.0 to 3.5%; from 3.5 to 4.0%; from 4.0 to 4.5%; from 4.5 to 5.0%; and in particular about 2.5%.

According to a particular embodiment, the subject of the present invention is an association as defined above, comprising at least one other substance having a soothing effect on the skin.

According to a particular embodiment, the subject of the present invention is an association as defined above, said substance having a soothing effect on the skin being chosen from acetyl hexapeptide-1, an oil containing α-linolenic or a natural extract of Tasmannia Lanceolata.

According to a particular embodiment, the subject of the present invention is an association as defined above, further comprising acetyl hexapeptide-1.

“Acetyl hexapeptide-1” is a hexapeptide of the sequence “Ac-Nle-Ala-His-(D)Phe-Arg-Trp-NH₂”. It is a biomimetic peptide (FR 2835528) of α-MSH. The structure of the molecule, whose CAS number is [448944-47-6], is shown above.

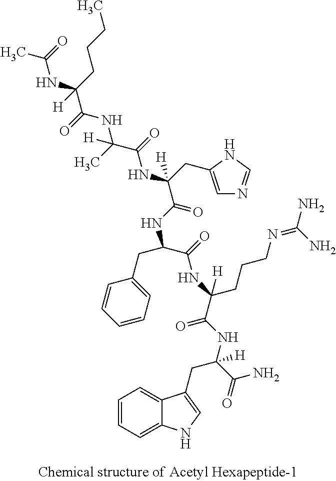 [SMILES: CCCC[C@H](NC(C)=O)C(=O)NC(C)C(=O)N[C@@H](Cc1cnc[nH]1)C(=O)N[C@H](Cc1ccccc1)C(=O)N[C@@H](CCCN=C(N)N)C(=O)N[C@H](Cc1c[nH]c2ccccc12)C(N)=O]

According to a particular embodiment, the acetyl hexapeptide-1 is present in an amount of 2 ppm to 10 ppm.

The term “from 2 ppm to 10 ppm” of acetyl hexapeptide-1 means the following ranges: from 2 ppm to 4 ppm; from 4 ppm to 6 ppm; from 6 ppm to 8 ppm; from 8 ppm to 10 ppm.

Acetyl hexapeptide-1 is marketed as Melitane™ (INCI name: Glycerin (and) Water (and) Dextran (and) Acetyl Hexapeptide-1). Melitane™ from Lucas Meyer Cosmetics is a solution of acetyl hexapeptide-1 in a mixture of glycerin, dextran and water.

The Melitane™ product from Lucas Meyer Cosmetics is advantageously used in an amount comprised from 0.5% to 5.0%, as a percentage by weight relative to the total weight, or from 0.5 to 1.0%; from 1.0 to 2.0%; from 2.0 to 3.0%; from 3.0 to 4.0%; from 4.0 to 5.0%; and in particular about 0.5%.

Acetyl hexapeptide-1 can come in hydrophilic or lipophilic versions. In oily form, it is also marketed under the name MelinOil™ (INCI name: Isopropyl Palmitate (and) Lecithin (and) Water (and) Acetyl Hexapeptide-1) by the company Lucas Meyer Cosmetics.

The MelinOil™ product from Lucas Meyer Cosmetics is advantageously used in an amount comprised from 0.5% to 5.0%, as a percentage by weight relative to the total weight, or from 0.5 to 1.0%; from 1.0 to 2.0%; from 2.0 to 3.0%; from 3.0 to 4.0%; from 4.0 to 5.0%.

According to a particular embodiment, the subject of the present invention is an association as defined above, comprising at least one oil containing α-linolenic acid.

α-linolenic acid (ALA) is an omega-3 polyunsaturated fatty acid. It is a carboxylic acid with a chain of 18 carbon atoms and three cis double bonds; the first of the double bonds is positioned on the third carbon atom counted from the end of the chain, noted ω. It is the main omega-3 fatty acid. Oils containing omega-3 also contain omega-6 that compete with omega-3 at the cellular level due to their opposed physiological effects.

According to a particular embodiment, the subject of the present invention is an association as defined above, comprising at least one oil containing a high level of α-linolenic acid.

By “high level of α-linolenic acid”, is meant within the meaning of the present invention, an oil containing at least 33% by weight, preferably from 33% to 66% by weight, of α-linolenic acid with respect to the total fatty acids of the oil.

According to a particular embodiment, the subject of the present invention is an association as defined above, comprising at least one oil containing α-linolenic acid with an omega-3/omega-6 ratio of 0.20 to 1.

According to a particular embodiment, the subject of the present invention is an association as defined above, comprising at least one oil containing α-linolenic acid with an α-linolenic acid content of 33% to 66% relative to the total fatty acids in the oil and with an omega-3/omega-6 ratio of 0.20 to 1.0.

According to a particular embodiment, the oil containing α-linolenic acid is a vegetable oil.

According to a particular embodiment, the subject of the present invention is an association as defined above, comprising at least one oil containing α-linolenic acid chosen from Perilla oil, linseed oil, camelina oil, Inca Inchi oil, chia oil, Rosehip oil from Chile or mixtures thereof.

Rosehip oil from Chile can be extracted from the seeds of the fruit of the rosebush, advantageously according to the process consisting in extracting the oil by cold pressing.

According to a particular embodiment, the subject of the present invention is an association as defined above, comprising a natural extract of Tasmannia Lanceolata.

Tasmannia lanceolata (also known as Drimys aromatica or Drimys lanceolata, and commonly known as Tasmanian pepper or mountain pepper) is a wild plant of the Winferaceae family, native to northern Tasmania. Tazman Pepper™ (INCI name—Glycerin (and) Water (and) Tasmannia Lanceolata Fruit/Leaf Extract) is marketed by Lucas Meyer Cosmetics.

Tazman Pepper™ is an extract of fruits and leaves from Tasmannia Lanceolata in a mixture of glycerin and water and can be advantageously used in the context of the present invention.

According to a particular embodiment, the subject of the present invention is an association according to the invention, further comprising acetyl hexapeptide-1, in particular from 2 ppm to 10 ppm, and/or comprising at least one oil containing α-linolenic acids,
in particular chosen from Perilla oil, linseed oil, camelina oil, Inca Inchi oil, Chia oil and Chilean rosehip oil,
in particular with an α-linolenic acid content of 33% to 66% relative to the total fatty acids of the oil and with an omega-3/omega-6 ratio of 0.20 to 1.0;
and/or comprising a natural extract of Tasmannia Lanceolata.

Another object of the present invention is the use of an association as defined above, as a cosmetic composition.

By “cosmetic composition” is meant any composition for cosmetic purposes, in particular a composition that can be brought into contact with the surface parts of the human body, the skin, in particular the epidermis, the mucous membranes and the scalp.

Within the meaning of the present invention, the term “cosmetic composition” is meant a non-pharmaceutical composition, that is to say which does not require therapeutic treatment, that is to say intended for any zone of healthy skin.

By “healthy skin” is meant an area of skin to which the association or composition according to the invention is applied, said to be “non-pathological” by a dermatologist, that is to say not showing any infection, disease, or sores or injuries and/or other dermatoses.

Another object of the present invention relates to a cosmetic composition, comprising an association as defined above, in a cosmetically acceptable medium.

By “a cosmetically acceptable medium”, is meant within the meaning of the invention a medium compatible with use in cosmetics.

According to a particular embodiment, the present invention relates to a cosmetic composition, comprising an association as defined above, said composition comprising at least one other ingredient. Said ingredient is chosen in particular from moisturizing agents, chemical filters, sun filters, in particular UV A filters, inorganic mineral sun filters, in particular titanium dioxide, thermal waters, in particular Treignac® water.

Among the “hydrating agents”, mention may be made, by way of example, of sodium lactate, polyols, in particular glycerin, propylene glycol, butylene glycol, pentylene glycol, hexylene glycol, dipropylene glycol, diethylene glycol mannitol and amino acids, caprylic/capric triglyceride, or a mixture of these agents.

As examples of sun filters that can be combined with the composition of the present invention as defined above, mention can be made of all those appearing in the modified cosmetics directive 76/768/EEC, as appendix VII.

### Examples of “Chemical Filters” Include:

anthranilates, such as menthyl anthranilate,
benzophenones, such as benzophenone-2 (oxybenzone), benzophenone-4 (Uvinul® MS40 (INCI: benzophenone-4))
benzylidene-camphors, such as 4-methylbenzylidene camphor (Eusolex® 6300 (INCI: Methylbenzylidene Camphor)),
benzimidazoles, such as benzimidazilate (Neo Heliopan® AP (INCI: Disodium Phenyl Bidenzimidazole Tetrasulfonate)), or phenylbenzimidazole sulphonic acid (Eusolex® 232 (INCI: Phenylbenzimidazole Sulfonic Acid)).
benzotriazoles, such as methylene bis-benzotriazolyltetramethylbutylphenol (Tinosorb® M (INCI: Methylene Bis-Benzotriazolyl Tetramethylbutylphenol (nano)),
cinnamates, such as ethocrylene (Uvinul® N35), octylmethoxycinnamate (Parsol® MCX(INCI: Ethylhexyl Methoxycinnamate), or octocrylene (Uvinul® 539 (INCI: Octocrylene)),
dibenzoylmethanes, such as butyl methoxydibenzoylmethane (Parsol® 1789 (INCI: Butyl Methoxydibenzoylmethane)),
imidazolines, such as ethylhexyl dimethoxybenzylidene dioxoimidazoline,
PABAs, such as diethylhexylbutamido-triazone (Uvasorb® HEB (INCI: Diethylhexyl butamido triazone)), ethylhexyltriazone (Uvinul® T150 (INCI: Ethylhexyl Triazone), or ethyl PABA (benzocaine),
salicylates, such as dipropylene glycol salicylate, ethylhexyl salicylate, homosalate, or TEA salicylate,
triazines, such as anisotriazine (Tinosorb® (INCI: Bis-Ethylhexyloxyphenol methoxyphenyl Triazine)),
   or a mixture of these filters.

Among the “inorganic filters”, also called “mineral screens”, which can be associated with the formulation for topical use which is the subject of the present invention, mention may be made of titanium oxides such as TiO₂, zinc oxides such as ZnO, cerium oxide, zirconium oxide, yellow, red or black iron oxides, chromium oxides, or a mixture of these filters. These mineral screens may or may not be micronized, may or may not have undergone surface treatments and may optionally be presented in the form of aqueous or oily pre-dispersions.

The composition of the present invention may also contain other adjuvants and excipients usual in the cosmetic fields, such as cosmetic oils, in particular oils containing antioxidants, preservatives, emulsifiers, hydrophilic or lipophilic gelling agents, hydrophilic active ingredients or lipophilic.

The term “cosmetic oils” refers to oils compatible with cosmetic use.

The “cosmetic oils” according to the present invention may be, or may contain, by way of example:
triglyceride-based vegetable oils such as sunflower oil, sesame oil, rapeseed oil, sweet almond oil, calophyllum oil, palm oil, avocado oil, jojoba oil, olive oil, castor oil or cereal germ oils such as wheat germ oil, apricot oil,
fatty acids such as α-linolenic acid, or containing α-linolenic acid,
esters of fatty acids and alcohols or polyols such as isopropyl, butyl or cetyl myristates, isopropyl, butyl or ethyl-2-hexyl palmitates, isopropyl, butyl, octyl, hexadecyl or isocetyl stearates, decyl oleate, hexyl laurate, esters derived from lanolic acid such as isopropyl or isocetyl lanolates, isononanoate isononyl, diisopropyl adipate, propylene glycol dicaprylate, glycol or glycerol octanoates and decanoates as well as cetyl ricinoleate,
   or a mixture of these oils.

As “antioxidants”, we can cite, for example, tocopherol (vitamin E) or ascorbic acid (vitamin C).

As “preservatives”, mention may be made, for example, of benzalkonium chloride, phenoxyethanol, or sorbic acid.

As “emulsifiers”, mention may be made, for example, of polyol fatty acid esters, for example glyceryl stearate, PEG-40 stearate, sorbitan tristearate, polyoxyethylene sorbitan stearates (Tween®-60 (INCI: Polysorbate 60) or Tween®-20 (INCI: Polysorbate 20)), ceteareth-20 (ethoxylated) (INCI: Ceteareth-20), cetyl alcohol.

As “hydrophilic gelling agent”, mention may be made, for example, of carboxyvinyl polymers, acrylic copolymers, polysaccharides, natural gums, such as xanthan gum, clays, Sepigel™ 305 (INCI: Polyacrylamide (AND) C13-14 Isoparaffin (AND) Laureth-7).

As a “lipophilic gelling agent” mention may be made of hydrophobic silica.

Said adjuvants and other active principles can be present in the composition in amounts conventionally used in cosmetics, in particular from 0.01 to 20% in percentage by weight relative to the total weight of the composition.

According to another particular embodiment, the present invention relates to a cosmetic composition as defined above, said cosmetic composition being formulated for topical application.

According to another particular embodiment, the present invention relates to a cosmetic composition as defined above, said composition being in the form of an emulsion, a cream, a gel, a dispersion, a serum, foam, body milk or anhydrous balm.

An emulsion according to the present invention can be an oil-in-water emulsion, or a water-in-oil emulsion.

The fats and emulsifiers present in the emulsions according to the present invention are those usually used in cosmetics.

The fats that can be used are, for example, mineral or vegetable oils.

The emulsifiers can in particular be chosen from polyol fatty acid esters, for example glyceryl stearate, PEG-40 stearate, sorbitan tristearate, stearates of polyoxyethylene sorbitan (Tween®-60 or Tween®-20), ceteareth-20 (ethoxylated).

According to another particular embodiment, the present invention relates to a cosmetic composition as defined above, said composition being in the form of a day cream.

According to another particular embodiment, the present invention relates to a cosmetic composition as defined above, said composition being in the form of a night cream.

According to another particular embodiment, the present invention relates to a cosmetic composition as defined above, said cosmetic composition being formulated for topical application, in particular topical application to the skin, said composition being in particular in the form of an emulsion, a cream, a gel, a dispersion, a serum, a mousse, a body milk or anhydrous balm, in particular in the form of a day cream or a night cream.

Another object of the present invention is the use of an association according to the invention as defined above, or of a cosmetic composition according to the invention as defined above, as an after-sun product.

According to another particular embodiment, the present invention relates to the cosmetic use of an association according to the invention as defined above, or of a cosmetic composition according to the invention as defined above, as an after-sun product.

Within the meaning of the present invention, the term “cosmetic use” is meant a non-pharmaceutical use, that is to say which does not require therapeutic treatment, that is to say intended for any zone of healthy skin.

Another subject of the present application is the association according to the invention as defined above or cosmetic composition according to the invention as defined above for its use as an after-sun product.

Another object of the present invention is the use of an association according to the invention as defined above, or of a cosmetic composition according to the invention as defined above, in the cosmetic treatment of healthy skins which are sensitive and/or reactive.

“Sensitive and/or reactive skin” means any skin subject to a feeling of discomfort that may manifest itself in more or less diffuse and localized redness, itching, tightness, irritation, burning sensations.

According to another particular embodiment, the present invention relates to the cosmetic use of an association according to the invention, or of a cosmetic composition according to the invention as defined above, in the cosmetic treatment of sensitive and/or or reactive skin.

Within the meaning of the present invention, the term “cosmetic treatment” is meant a non-therapeutic treatment, that is to say intended for any zone of healthy skin.

According to another particular embodiment, the present invention relates to the cosmetic use of an association according to the invention, or of a cosmetic composition according to the invention, to prevent and/or reduce the sensations of discomfort resulting from sensitive or reactive skin, and in particular the sensations of itching, pruritus, tingling, pins and needles, itching or tightness.

According to another particular embodiment, the present invention relates to the cosmetic use of an association according to the invention as defined above, or of a cosmetic composition according to the invention as defined above, which can be administered by topical to prevent or slow down the appearance of dysesthetic sensations on sensitive and/or reactive human skin.

By “dysesthetic sensations” is meant within the meaning of the present application sensations felt in a skin area such as tingling, tingling, itching, burning, heating or tightness.

Another object of the present application is the association according to the invention as defined above or the cosmetic composition according to the invention as defined above for its use in the treatment of skin which is sensitive and/or reactive.

According to another particular embodiment, the present invention relates to the association according to the invention as defined above or the cosmetic composition according to the invention as defined above for its use in preventing and/or reducing the sensations of discomfort resulting from sensitive or reactive skin, and in particular the sensations of itching, pruritus, tingling, tingling, itching or tightness. According to another particular embodiment, the present invention relates to the association according to the invention as defined above or the cosmetic composition according to the invention as defined above for its use, which can be administered topically, to prevent or slow down the appearance of dysesthetic sensations on sensitive and/or reactive human skin.

Another object of the present invention is a method for the cosmetic treatment of sensitive skins, comprising the topical application to healthy skin of an association according to the invention as defined above, or of a cosmetic composition according to invention as defined above to reduce the sensations of tingling, tingling, itching or pruritus, burning, heating, discomfort or tightness of the skin.

According to a particular embodiment, the present invention relates to a method for the cosmetic treatment of sensitive skin, comprising the topical application to healthy skin of an association according to the invention as defined above, or one of a cosmetic composition according to one as defined above to reduce sensations of stinging, tingling or discomfort of the skin.

Within the meaning of the present invention, the term “cosmetic treatment method” means a method which does not require therapeutic treatment, that is to say a treatment method intended for any zone of healthy skin.

According to another particular embodiment, the present invention relates to a method for the cosmetic treatment of sensitive skins according to the invention, comprising an application from 1 to 3 times a day, in particular 1 time in the morning and 1 time in the evening on healthy skin.

According to another particular embodiment, the present invention relates to a method for the cosmetic treatment of erythema and inflammation of the skin due to exposure to the sun, comprising the application to the skin of an association according to the invention, or of a cosmetic composition according to the invention.

Another object of the present invention is the use of an association according to as defined above, as a pharmaceutical composition, in particular dermatological.

By “pharmaceutical composition” is meant any composition for pharmaceutical purposes, in particular a dermatological composition which can be brought into contact with the superficial parts of the human body, the skin, in particular the epidermis.

Another object of the present invention is the association according to the invention as defined above, for its use as a medicament, in particular as a dermatological medicament.

According to another particular embodiment, the present invention relates to the association according to the invention as defined above for its use for reducing the sensations of itching or pruritus, burning, heating or tightness of the skin.

Another object of the present invention is a dermatological composition, comprising, as active substance, an association as defined above, with a pharmaceutically acceptable excipient.

By “pharmaceutically acceptable excipient” is meant within the meaning of the invention any compound making it possible to facilitate the shaping of the composition and not modifying the nature of the biological activity of the active principle, with a pharmaceutical use. A pharmaceutically acceptable excipient can be, by way of example, a solvent, plasticizer, lubricant, dispersing medium, agents delaying absorption, flow agent.

According to another particular embodiment, the subject of the present invention is a dermatological composition as defined above, said dermatological composition being formulated for topical application, in particular in the form of an emulsion, a cream, a gel, a dispersion, a serum, a mousse, a body milk or an anhydrous balm.

Another object of the present invention is a dermatological composition as defined above, for its use for treating or preventing pruriginous inflammatory dermatoses.

Examples of formulas according to the invention are given by way of non-limiting examples.

The inventors have developed an oil-in-water emulsion with the proportions indicated in Table 1. Said composition comprises a mixture of cannabidiol and palmitoyl tripeptide-8 (Neutrazen™). The composition of Table 1 can be used both as a cosmetic composition or a dermatological composition.

TABLE 1
Composition of an emulsion

| Phase | Ingredient | w/w % |
|---|---|---|
| A | Emulsifier1 | 1.50 |
| | Emulsifier2 | 1.50 |
| | Emollient | 15.00 |
| B | Solvent | QSP 100 |
| | Thickening agent | 1.00 |
| | Neutrazen ™ | 2.50 |
| | Cannabidiol | 0.50 |
| | Conservative | 0.30 |

Another composition developed by the inventors is a composition with the proportions indicated in Table 2. Said composition comprises a mixture of cannabidiol and palmitoyl tripeptide-8 (Neutrazen™) and an oil containing α-linolenic acids. It is a cream-gel composition that can be used both as a cosmetic composition or a dermatological composition.

TABLE 2
Composition of a cream-gel

| Phase | Ingredient | w/w % |
|---|---|---|
| A | Solvent | QSP 100 |
| | Thickening agent | 0.60 |
| B | Fixing agent | 5.00 |
| | Conservative | 0.30 |
| C | Consistency agent | 5.00 |
| | Emollient 2 | 2.00 |
| | Emollient 3 | 3.00 |
| | Mineral oil | 2.00 |
| | Perilla oil | 2.00 |
| | Cannabidiol | 0.50 |
| D | Emulsifier | 0.70 |
| | Solvent | 20.00 |
| | Neutrazen ™ | 2.50 |

Another composition developed by the inventors is a composition with the proportions indicated in Table 3.

Said composition includes a mixture of cannabidiol and palmitoyl tripeptide-8 (Neutrazen™), an extract of Tasmannia Lanceolata (Tazman Pepper™) and acetyl hexapeptide-1 (Melitane™). It is a cream-gel composition that can be used both as a cosmetic composition or a dermatological composition.

TABLE 3
Composition of a cream-gel

| Phase | Ingredient | w/w % |
|---|---|---|
| A | Solvent | QSP 100 |
| | Conservative 1 | 0.30 |
| | Conservative 2 | 0.60 |
| | Chelating agent | 0.10 |
| B | Gelling agent | 1.75 |
| C | Emollient 1 | 3.00 |
| | Emollient 2 | 4.00 |
| | Emollient 3 | 3.00 |
| | Antioxidant | 0.20 |
| D | Tazman Pepper ™ | 2.00 |
| | Neutrazen ™ | 2.50 |
| | Cannabidiol | 0.50 |
| | Melitane ™ | 0.50 |

### LEGENDS OF FIGS.

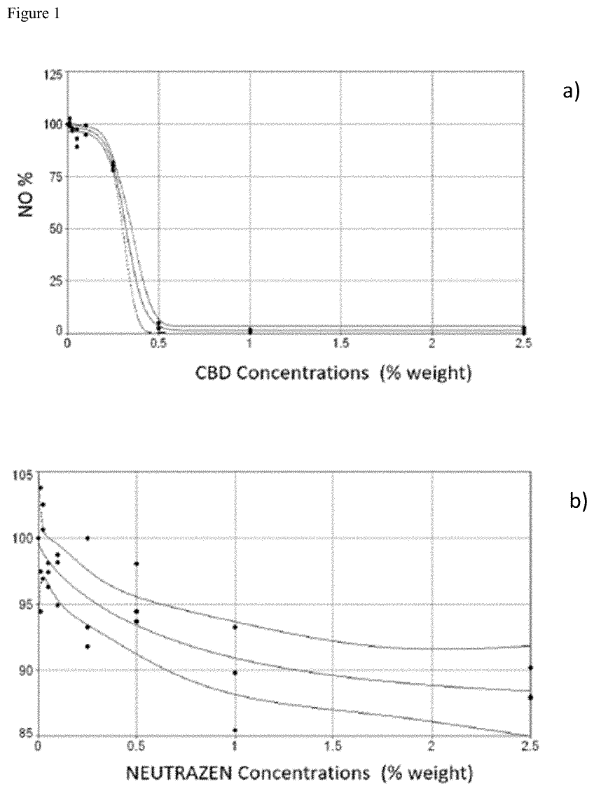

FIG. 1 represents the dose-response curves of the inhibition of NO production by nonlinear regression of 3 series of experiments, part a) for different concentrations of cannabidiol and part b) for different concentrations of Neutrazen™

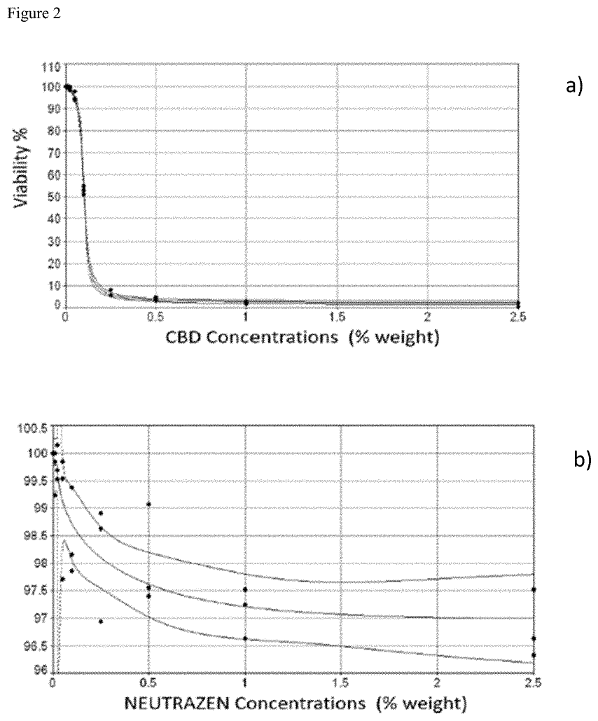

FIG. 2 shows the dose-response curves of cell viability by non-linear regression of 3 series of experiments, part a) for different concentrations of cannabidiol and part b) for different concentrations of Neutrazen™.

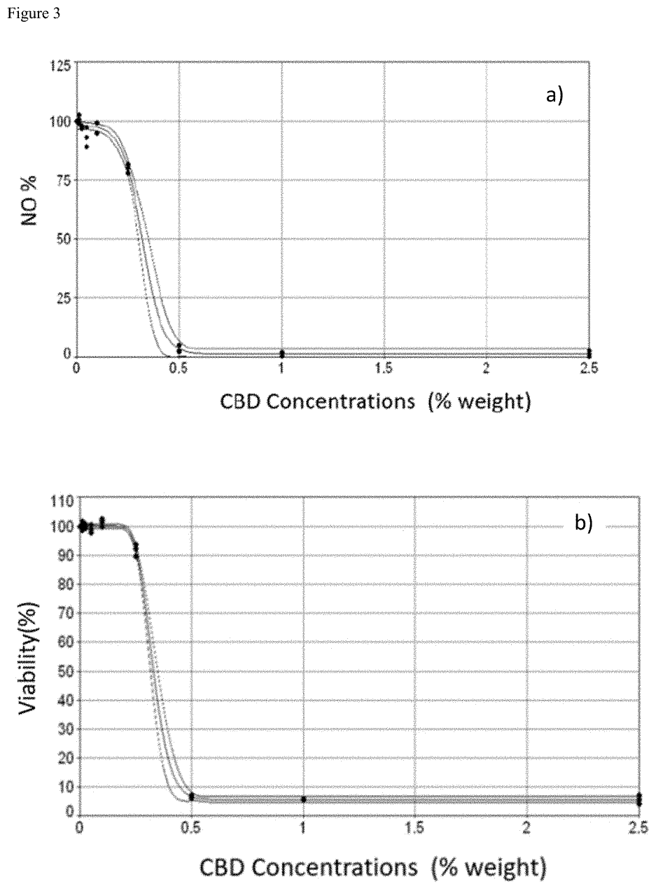

FIG. 3 represents for mixtures containing 0.01% of Neutrazen™ and different concentrations of CBD (0.01, 0.025, 0.05, 0.1, 0.25, 0.5, 1 and 2.5%), in part a) the dose-response of the inhibition of NO production and partly b) those of cell viability, the curves being obtained by non-linear regression of 3 series of experiments.

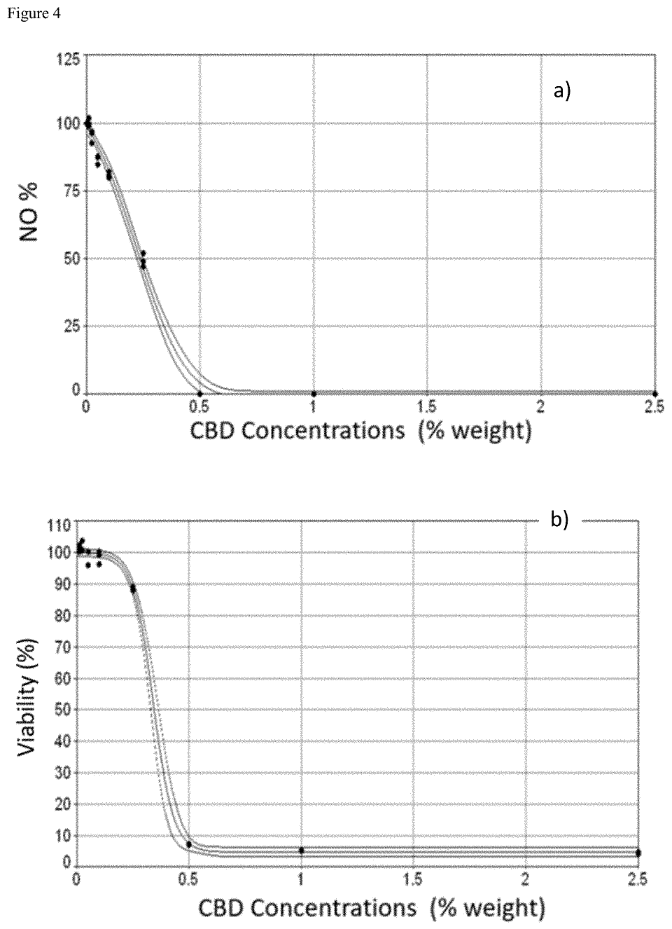

FIG. 4 represents for the mixtures containing 0.025% of Neutrazen™ and different concentrations of CBD (0.01, 0.025, 0.05, 0.1, 0.25, 0.5, 1 and 2.5%), in part a) the dose-response curves of the inhibition of NO production and in part b) those of cell viability, the curves being obtained by non-linear regression of 3 series of experiments.

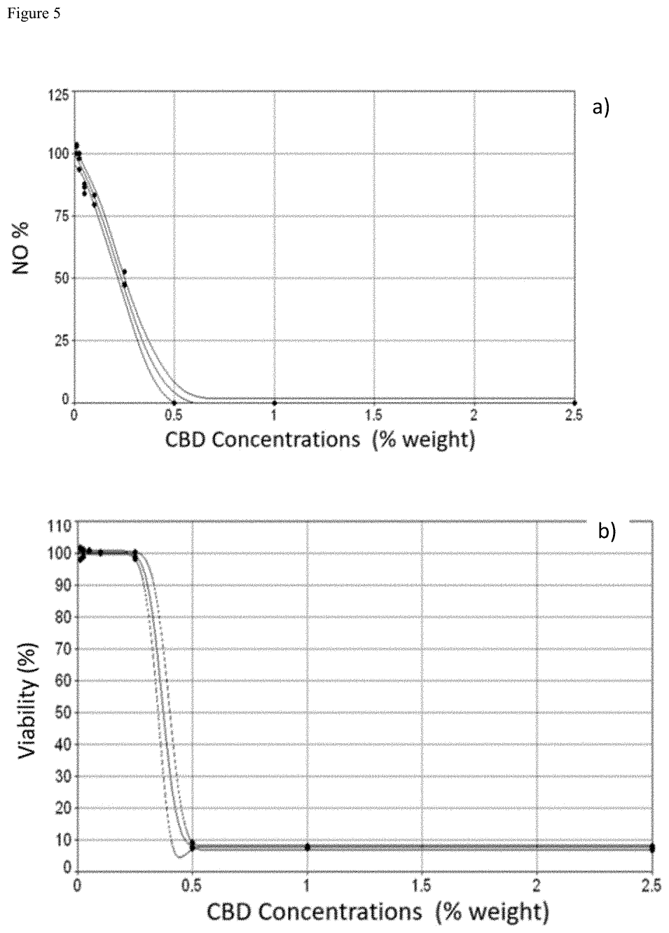

FIG. 5 represents for the mixtures containing 0.05% of Neutrazen™ and different concentrations of CBD (0.01, 0.025, 0.05, 0.1, 0.25, 0.5, 1 and 2.5%), in part a) the dose-response curves of the inhibition of NO production and in part b) those of cell viability, the curves being obtained by non-linear regression of 3 series of experiments.

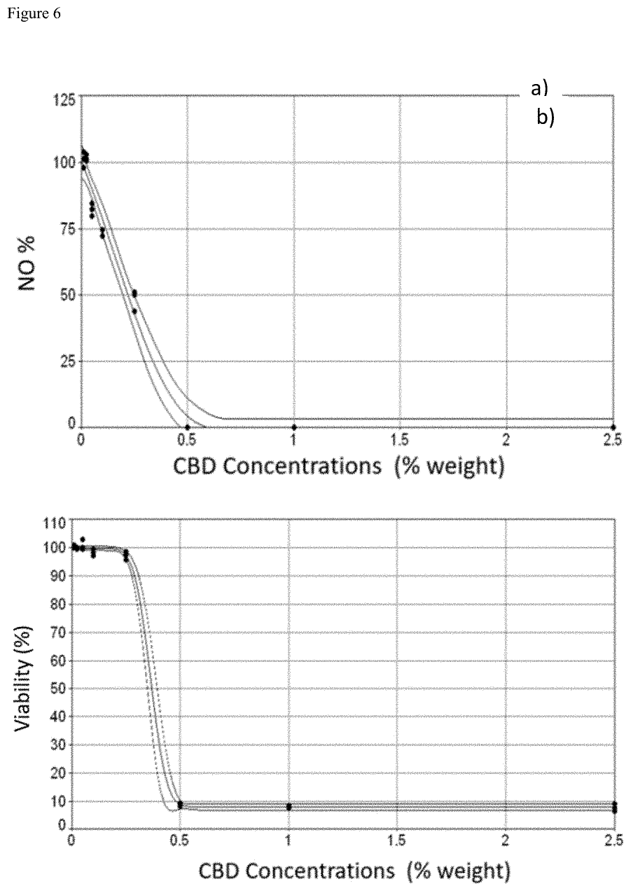

FIG. 6 represents for the mixtures containing 0.01% of Neutrazen™ and different concentrations of CBD (0.01, 0.025, 0.05, 0.1, 0.25, 0.5, 1 and 2.5%), in part a) the dose-response curves of the inhibition of NO production and partly b) those of cell viability by non-linear regression of 3 series of experiments.

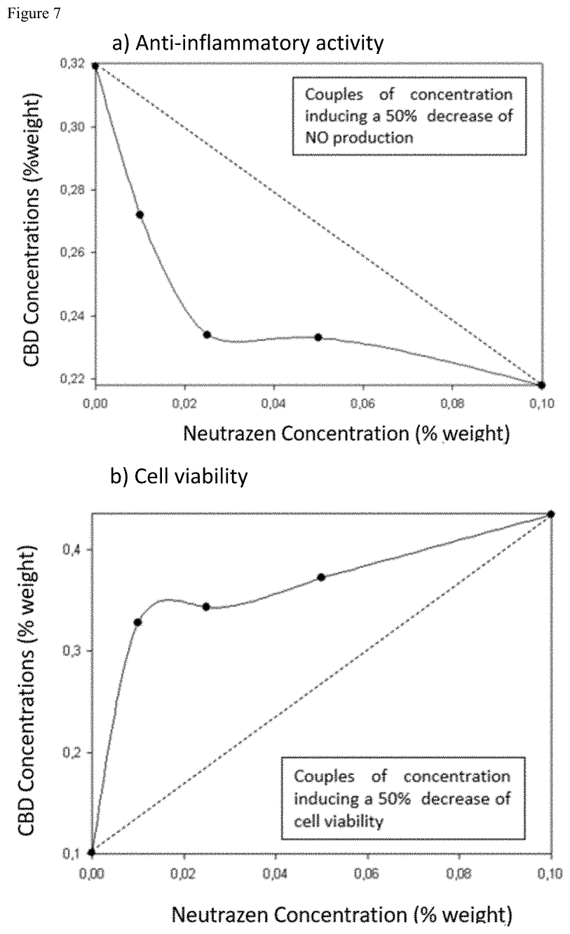

FIG. 7 reports the isobolograms, in part a) for inflammatory activity and in part b) for cell viability; in the figures the theoretical line observed for the additive effects is represented by a dotted line. The following examples illustrate the invention, without limiting its scope.

### EXAMPLE 1

### Formulation of an Emulsion

TABLE 4
Formulation of an emulsion

| | | | w/w |
|---|---|---|---|
| Phase | Ingredient | INCI | % |
| A | Montanov ™68 | Cetearyl Alcohol (and) Cetearyl | 1.50 |
| | | Glucoside | |
| | Montanov ™202 | Arachidyl Alcohol (and) Behenyl | 1.50 |
| | | Alcohol (and) Arachidyl Glucoside | |
| | Lanol ™1688 | Cetearyl Ethylhexanoate | 15.00 |
| B | Water | Water | 77.70 |
| | Simugel ™ EG | Sodium Acrylate/Acryloyldimeth- | 1.00 |
| | | ylaurate Copolymer (and) | |
| | | Isohexadecane (and) Polysorbate 80 | |
| | Neutrazen ™ | Water (and) Butylene Glycol (and) | 2.50 |
| | | Dextran (and) Palmitoyl Tripeptide-8 | |
| | Cannabidiol | Cannabidiol | 0.50 |
| | Sepicide ™ HB | Phenoxyethanol (and) Methylparaben | 0.30 |
| | | (and) Ethylparaben (and) Propylparaben | |
| | | (and) Butylparaben | |

### EXAMPLE 2

### Cream Gel Containing an α-Linolenic Oil

TABLE 5
Formulation of a Cream gel containing an α-linolenic oil

| | | | w/w |
|---|---|---|---|
| Phase | Ingredient | INCI | % |
| A | Water | Water | 58.88 |
| | Carbopol ® Ultrez-21 | Acrylate/C10-30 Alkyl Acrylate | 0.60 |
| | | Crosspolymer | |
| B | Polyethylene Glycol 400 | PEG-8 | 5.00 |
| | Sepicide ™ HB | Phenoxyethanol (and) | 0.30 |
| | | Methylparaben (and) | |
| | | Ethylparaben (and) | |
| | | Propylparaben (and) | |
| | | Butylparaben | |
| C | Cutina ® MD | Glyceryl Stearate | 5.00 |
| | Cetearyl Alcohol | Cetearyl Alcohol | 2.00 |
| | Cetiol ® CC | Carbonate Dicaprylyl | 3.00 |
| | Mineral Oil USP | Mineral Oil | 2.00 |
| | Perilla oil | Perilla Ocymoides Seed Oil | 2.00 |
| | Cannabidiol | Cannabidiol | 0.50 |
| D | Parfum | Fragance | 0.02 |
| | Triethanolamine 99 | Triethanolamine | 0.70 |
| | Water | Water | 20.00 |
| | Neutrazen ™ | Water (and) Butylene Glycol | 2.50 |
| | | (and) Dextran (and) Palmitoyl | |
| | | Tripeptide-8 | |

### EXAMPLE 3

### Composition Containing Acetyl Hexapeptide-1 and an Extract of Tasmannia Lanceolata

TABLE 6
composition containing acetyl hexapeptide-1
and an extract of Tasmannia Lanceolata

| | | | w/w |
|---|---|---|---|
| Phase | Ingredient | INCI | % |
| A | Water | Water | 80.35 |
| | Chlorphenesin | Chlorphenesin | 0.30 |
| | Phenoxyethanol | Phenoxyethanol | 0.60 |
| | Dermofeel ™PA-3 | Sodium Phytate (and) Water | 0.10 |
| | | (and) Alcohol | |
| B | Lecigel ™ | Sodium Acrylate Copolymer | 1.75 |
| | | (and) Lecithin | |
| C | Dermofeel ™ BGC | Butylene Glycol Dicaprylate | 3.00 |
| | | (and) Dicaprate | |
| | DUB ININ | Isononyl Isononanoate | 5.00 |
| | Myritol ® 318 | Caprylic (and) Capric Triglyceride | 3.00 |
| | Vitapherole ® E1000 | Tocopherol (and) Helianthus | 0.20 |
| | | Annuus (Sunflower) Seed Oil | |
| D | Tazman Pepper ™ | Glycerin (and) Water (and) | 2.00 |
| | | Tasmannia Lanceolata Fruit/ | |
| | | Leaf Extract | |
| | Neutrazen ™ | Water (and) Butylene Glycol | 2.50 |
| | | (and) Dextran (and) Palmitoyl | |
| | | Tripeptide-8 | |
| | Cannabidiol | Cannabidiol | 0.50 |
| | Melitane ™ | Glycerin (and) Water (and) | 0.50 |
| | | Dextran (and) Acetyl | |
| | | Hexapeptide-1 | |

### EXAMPLE 4

In-vitro analysis of the anti-inflammatory activity of the cannabidiol/palmitoyl tripeptide-8 association on murine macrophages.

### I. Aim of the Study

Low-grade inflammation is strongly implicated in degenerative processes in human skin such as photoaging and atopy. The reduction of low-intensity inflammatory reactions by topical products may be necessary in the event of cutaneous aggression, to obtain optimal healing and restore the physiological balance of human skin. In order to identify new anti-inflammatory candidates, an in vitro anti-inflammatory test was developed with a murine cell line: the murine macrophage test RAW 267.4. This test was used to monitor the inhibitory effects of chemical compounds or natural extracts on the low-intensity inflammatory cascade leading to overproduction of nitrogen oxide (NO) in the endothelial wall of blood vessels.

The study was carried out to evaluate the ability of cannabidiol, palmitoyl tripeptide-8 (active ingredient in Neutrazen™), and their mixture to inhibit the pro-inflammatory cascade leading to the production of NO in murine macrophages stimulated by liposaccharides (LPS).

### II. Materials and Methods.

### Materials

Cannabidiol is used in the form of a white powder, for example marketed by PHYTOGRASE.

A Neutrazen™ solution containing palmitoyl tripeptide-8 is used.

Neutrazen™ comes in the form of a translucent liquid. It is marketed by the Lucas Meyer company. It is composed of water, butylene glycol, dextran and palmitoyl tripeptide-8.

The INCI name for Neutrazen™ is: Water (and) Butylene Glycol (and) Dextran (and) Palmitoyl Tripeptide-8.

### Principle of the Tests

The in vitro anti-inflammatory test is based on the ability of murine macrophages to generate a strong inflammatory response when stimulated by antigens such as LPS. Immobilized murine macrophages (RAW 267.4 cell line) are stimulated with LPS from E. coli and exposed for 24 hours. At the end of the incubation period, the production of NO is evaluated indirectly by measuring the accumulation of nitrite/nitrate, stable final products of the oxidation of NO, in the culture medium using a method spectrophotometric based on the Griess reaction.

### Cell Line

MURINS RAW 267.4 Macrophage cell line (Sigma-Aldrich, No. P6110401, Lot. 091006), low number of passages (less than 50).

### Culture Medium

Complete medium: DMEM with stabilized L-glutamine (Dulbecco's Minimum Essential Medium, PAN BIOTECH. Lot 974251) supplemented with Penicillin 100 IU/ml and streptomycin 100 μg/m1 (PAN BIOTECH, Lot 225614), and 10% inactivated calf serum (PAN BIOTECH, Lot P460518), pH 7.2, freshly prepared, stored for less than 3 weeks.

### Dilution of Materials

Cannabidiol (CBD) is diluted in DMSO (10 mg/mL storage solution), and subjected to an ultrasonic bath for 20 minutes. The Neutrazen™ solution is diluted in PBS (Phosphate buffer saline, Sigma-Aldrich).

### Controls

The negative control: DMSO 1% The positive control: Dexamethasone (Sigma-Aldrich) 1-5-10-50-100 μM.

### Test Procedure

RAW 267.4 murine macrophages were inoculated into 48-well tissue culture plates at a concentration of 1.10⁵ cells/ml (200 μl/well) and incubated for 24 hours at 37° C. (5% CO₂).

At the end of the incubation period, the culture medium was replaced with 200 μl of medium containing the appropriate concentrations of the material to be tested, and the cells were incubated at 37° C. (5% CO₂) for one hour.

At the end of the incubation period, pro-inflammatory E. coli LPS was added to the cell cultures (1 μg/ml. Then the cells were incubated at 37° C. (5% CO₂) for 24 hours.

### Evaluation of the Level of NO Released

The amount of NO released was measured in the culture supernatant by the Griess reaction. 100 μl of the supernatants were transferred into the wells of a 96-well tissue culture plate, and modified Griess reagent (SIGMA-ALDRICH) were added to each well. After a period of 15 min at room temperature, the Optical Density (OD) of each well was read at a wavelength of 540 nm by an Infinite M200 Pro fluorescence-luminescence reader (TECAN). The results obtained for the wells treated with the test material were compared with those of the untreated control wells (PBS, 100% NO production) and converted into percentage values.

### Evaluation of the Cell Viability

In parallel with the evaluation of the release of NO, the cell viability was measured to validate the tests. Vital Stain Reagent WST-1 was used to measure cellular mitochondrial respiration. For this, the culture medium was decanted and 100 μl of WST-1 reagent ( 1/10 dilution) were added to each well. After an incubation period of 30 min at 37° C. (5% CO2), the Optical Density (OD) of each well was read at a wavelength of 450 nm by an Infinite M200 Pro fluorescence-luminescence reader (TECAN). The results obtained for the wells treated with the test material were compared with those of the untreated control wells (DMSO, 100% viability) and converted into percentage values.

### Calculation of the IC50 for NO Release and the IC50 for Cell Viability

Inhibition of NO release and inhibition of cell viability were expressed as a percentage relative to the negative controls:

$$Percentage of NO release = \frac{100 \times ( test O D - control O D )}{DMSO control O D - control O D}$$

$$Percentage of cell viability = \frac{100 \times ( test O D - control O D )}{DMSO control O D - control O D}$$

The concentrations of test material causing a 50% decrease in NO release (IC50 (NO release)) and a 50% decrease in cell viability (IC50 (cell viability)) respectively were calculated using TableCurve software Version 2.0.

The anti-inflammatory ratio corresponds to the ratio between anti-inflammatory activity and toxicity. It is expressed as follows:

$$Anti - inflammatory ratio = \frac{IC 50 ( cell viability )}{IC 50 ( NO release )}$$

### EXAMPLE 5

Analysis of the anti-inflammatory activities of cannabidiol and Neutrazen™ (containing the palmitoy tripeptide-8)

The results concerning the anti-inflammatory activity of cannabidiol and Neutrazen™ (containing the palmitoy tripeptide -8) are reported in Table 7. FIG. 1 represents the dose-response curves of the inhibition of NO production by nonlinear regression of 3 series of experiments for different concentrations of cannabidiol (figure la) and for different concentrations of Neutrazen™ (FIG. 1b).

Results for macrophage viability of murine cannabidiol and Neutrazen™ (containing Palmitoy tripeptide-8) are reported in Table 8.

FIG. 2 shows the dose-response curves of cell viability by nonlinear regression of 3 series of experiments for different concentrations of cannabidiol (FIG. 2a) and for different concentrations of Neutrazen™ (FIG. 2b).

The results of the nonlinear regression analyzes to determine the IC50s and the anti-inflammatory ratio are reported in Table 9.

TABLE 7
Anti-inflammatory activity of cannabidiol and Neutrazen ™ (containing
palmitoyl-tripeptide-8)

| | Concentrations | Optical Densities observed for | | | NO production (% as compared | | |
|---|---|---|---|---|---|---|---|
| Samples | (weight %) | the 3 sets of experiments | | | to the negative control) | | |
| Blank | | 0.061 | 0.065 | 0.063 | — | — | — |
| Non-stimulated control | | 0.097 | 0.095 | 0.096 | — | — | — |
| Stimulated control | | 0.260 | 0.252 | 0.254 | 100 | 100 | 100 |
| CBD | 0.01% | 0.259 | 0.253 | 0.258 | 99.39 | 100.64 | 102.53 |
| | 0.025% | 0.255 | 0.248 | 0.251 | 96.93 | 97.45 | 98.10 |
| | 0.05% | 0.256 | 0.235 | 0.243 | 97.55 | 89.17 | 93.04 |
| | 0.1% | 0.252 | 0.251 | 0.253 | 95.09 | 99.36 | 99.37 |
| | 0.25% | 0.224 | 0.221 | 0.225 | 77.91 | 80.25 | 81.65 |
| | 0.5% | 0.101 | 0.103 | 0.1 | 2.45 | 5.10 | 2.53 |
| | 1% | 0.098 | 0.098 | 0.099 | 0.61 | 1.91 | 1.90 |
| | 2.5% | 0.097 | 0.099 | 0.098 | 0.00 | 2.55 | 1.27 |
| NeutraZen | 0.01% | 0.251 | 0.258 | 0.25 | 94.48 | 103.82 | 97.47 |
| | 0.025% | 0.255 | 0.256 | 0.255 | 96.93 | 102.55 | 100.63 |
| | 0.05% | 0.254 | 0.248 | 0.251 | 96.32 | 97.45 | 98.10 |
| | 0.1% | 0.257 | 0.25 | 0.246 | 98.16 | 98.73 | 94.94 |
| | 0.25% | 0.249 | 0.252 | 0.241 | 93.25 | 100.00 | 91.77 |
| | 0.5% | 0.251 | 0.249 | 0.244 | 94.48 | 98.09 | 93.67 |
| | 1% | 0.249 | 0.236 | 0.231 | 93.25 | 89.81 | 85.44 |
| | 2.5% | 0.244 | 0.233 | 0.235 | 90.18 | 87.90 | 87.97 |

TABLE 8
Cell Viability of cannabidiol and Neutrazen ™ (containing palmitoyl-tripeptide-8)

| | Concentrations | Optical Densities observed for | | | Cell viability (% as compared | | |
|---|---|---|---|---|---|---|---|
| Samples | (weight %) | the 3 sets of experiments | | | to the negative control) | | |
| Blank | | 0.07 | 0.07 | 0.071 | — | — | — |
| Non-stimulated control | | 0.075 | 0.071 | 0.075 | — | — | — |
| Stimulated control | | 0.730 | 0.726 | 0.721 | 100 | 100 | 100 |
| CBD | 0.01% | 0.726 | 0.724 | 0.722 | 99.39 | 99.69 | 100.15 |
| | 0.025% | 0.719 | 0.725 | 0.716 | 98.32 | 99.85 | 99.23 |
| | 0.05% | 0.716 | 0.690 | 0.681 | 97.86 | 94.50 | 93.81 |
| | 0.1% | 0.423 | 0.406 | 0.431 | 53.13 | 51.15 | 55.11 |
| | 0.25% | 0.129 | 0.108 | 0.112 | 8.24 | 5.65 | 5.73 |
| | 0.5% | 0.106 | 0.097 | 0.096 | 4.73 | 3.97 | 3.25 |
| | 1% | 0.094 | 0.084 | 0.087 | 2.90 | 1.98 | 1.86 |
| | 2.5% | 0.088 | 0.085 | 0.078 | 1.98 | 2.14 | 0.46 |
| NeutraZen | 0.01% | 0.725 | 0.726 | 0.720 | 99.24 | 100.00 | 99.85 |
| | 0.025% | 0.728 | 0.727 | 0.718 | 99.69 | 100.15 | 99.54 |
| | 0.05% | 0.715 | 0.723 | 0.720 | 97.71 | 99.54 | 99.85 |
| | 0.1% | 0.718 | 0.712 | 0.717 | 98.17 | 97.86 | 99.38 |
| | 0.25% | 0.721 | 0.706 | 0.714 | 98.63 | 96.95 | 98.92 |
| | 0.5% | 0.713 | 0.710 | 0.715 | 97.40 | 97.56 | 99.07 |
| | 1% | 0.708 | 0.708 | 0.705 | 96.64 | 97.25 | 97.52 |
| | 2.5% | 0.706 | 0.704 | 0.705 | 96.34 | 96.64 | 97.52 |

TABLE 9
Results of non-linear regression analysis

| | | | Anti- |
|---|---|---|---|
| | | | inflammatory |
| Samples | IC_{50-NO release} | IC_{50-cell viability} | ratio |
| Dexamethasone | 2.98 ± 0.18 μM | 192.65 ± 4.25 μM | 64.65 |
| CBD | 0.319 ± 0.006% | 0.101 ± 0.006% | <1 |
| NeutraZen | >2.5% | >2.5% | — |

### EXAMPLE 6

Analysis of the anti-inflammatory activity and of the cell viability of the cannabidiol/palmitoyl tripeptide-8 (Neutrazen™) association in a mixture by isobologram analysis. For each concentration of Neutrazen™ (0.01, 0.025, 0.05 and 0.1%), different concentrations of CBD are tested (0.01, 0.025, 0.05, 0.1, 0.25, 0.5, 1 and 2.5%) to determine both the production of NO and cell viability.

The results of the anti-inflammatory activity and cell viability assays are reported in Tables 10-13 and FIGS. 3-6.

Tests on anti-inflammatory activity and cell viability for mixtures containing 0.01% Neutrazen™ and different concentrations of CBD (0.01, 0.025, 0.05, 0.1, 0.25, 0.5, 1 and 2.5%) are reported in the table 10. FIG. 3 represents for the mixtures containing 0.01% of Neutrazen™ and different concentrations of CBD (0.01, 0.025, 0.05, 0.1, 0.25, 0.5, 1 and 2.5%), the dose-response curves of the inhibition of the production of NO (FIG. 3a) and cell viability (FIG. 3b) by nonlinear regression of 3 sets of experiments.

Tests on anti-inflammatory activity and cell viability for mixtures containing 0.025% Neutrazen™ and different concentrations of CBD (0.01, 0.025, 0.05, 0.1, 0.25, 0.5, 1 and 2.5%) are reported in the table 11. FIG. 4 represents for the mixtures containing 0.025% of Neutrazen™ and different concentrations of CBD (0.01, 0.025, 0.05, 0.1, 0.25, 0.5, 1 and 2.5%), the dose-response curves of the inhibition of the production of NO (FIG. 4a) and cell viability (FIG. 4b) by nonlinear regression of 3 sets of experiments.

Tests on anti-inflammatory activity and cell viability for mixtures containing 0.05% Neutrazen™ and different concentrations of CBD (0.01, 0.025, 0.05, 0.1, 0.25, 0.5, 1 and 2.5%) are reported in the table 12. FIG. 5 represents for the mixtures containing 0 05% of Neutrazen™ and different concentrations of CBD (0.01, 0.025, 0.05, 0.1, 0.25, 0.5, 1 and 2.5%), the dose-response curves of the inhibition of the production of NO (FIG. 5a) and cell viability (FIG. 5b) by nonlinear regression of 3 series of experiments.

Tests on anti-inflammatory activity and cell viability for mixtures containing 0.01% Neutrazen™ and different concentrations of CBD (0.01, 0.025, 0.05, 0.1, 0.25, 0.5, 1 and 2.5%) are reported in the table 13. FIG. 6 represents for the mixtures containing 0.01% of Neutrazen™ and different concentrations of CBD (0.01, 0.025, 0.05, 0.1, 0.25, 0.5, 1 and 2.5%), the dose-response curves of the inhibition of the production of NO (FIG. 6a) and cell viability (FIG. 6b) by nonlinear regression of 3 sets of experiments.

TABLE 10
Anti-inflammatory activity and cell viability
for mixture of NeutraZen 0.01% and cannabidiol.

| | Concentrations | Optical Densities observed for | | | NO production (% as compared | | |
|---|---|---|---|---|---|---|---|
| Samples | (weight %) | the 3 sets of experiments | | | to the negative control) | | |
| Blank | | 0.061 | 0.065 | 0.063 | — | — | — |
| Non-stimulated control | | 0.097 | 0.095 | 0.096 | — | — | — |
| Stimulated control | | 0.260 | 0.252 | 0.254 | 100 | 100 | 100 |
| CBD | 0.01% | 0.251 | 0.250 | 0.255 | 94.48 | 98.73 | 100.63 |
| | 0.025% | 0.239 | 0.235 | 0.242 | 87.12 | 89.17 | 92.41 |
| | 0.05% | 0.235 | 0.230 | 0.236 | 84.66 | 85.99 | 88.61 |
| | 0.1% | 0.228 | 0.216 | 0.231 | 80.37 | 77.07 | 85.44 |
| | 0.25% | 0.184 | 0.191 | 0.192 | 53.37 | 61.15 | 60.76 |
| | 0.5% | 0.089 | 0.091 | 0.097 | 0 | 0 | 0.63 |
| | 1% | 0.085 | 0.089 | 0.093 | 0 | 0 | 0 |
| | 2.5% | 0.089 | 0.089 | 0.089 | 0 | 0 | 0 |

| | | | |
|---|---|---|---|
| | Concentrations | Optical Densities observed for | Cell viability (% as compared |
| Samples | (weight %) | the 3 sets of experiments | to the negative control) |

| Blank | | 0.07 | 0.07 | 0.071 | — | — | — |
|---|---|---|---|---|---|---|---|
| Non-stimulated control | | 0.075 | 0.071 | 0.075 | — | — | — |
| Stimulated control | | 0.730 | 0.726 | 0.721 | 100 | 100 | 100 |
| CBD | 0.01% | 0.721 | 0.723 | 0.731 | 98.63 | 99.54 | 101.55 |
| | 0.025% | 0.728 | 0.721 | 0.726 | 99.68 | 99.24 | 100.77 |
| | 0.05% | 0.715 | 0.719 | 0.725 | 97.73 | 98.93 | 100.62 |
| | 0.1% | 0.741 | 0.726 | 0.738 | 101.66 | 100.00 | 102.63 |
| | 0.25% | 0.679 | 0.659 | 0.681 | 92.20 | 89.77 | 93.81 |
| | 0.5% | 0.116 | 0.118 | 0.119 | 6.34 | 7.18 | 6.81 |
| | 1% | 0.112 | 0.110 | 0.111 | 5.66 | 5.95 | 5.57 |
| | 2.5% | 0.122 | 0.108 | 0.103 | 7.21 | 5.65 | 4.33 |

TABLE 11
Anti-inflammatory activity and cell viability
for mixture of NeutraZen 0.025% and cannabidiol

| | Concentrations | Optical Densities observed for | | | NO production (% as compared | | |
|---|---|---|---|---|---|---|---|
| Samples | (weight %) | the 3 sets of experiments | | | to the negative control) | | |
| Blank | | 0.061 | 0.065 | 0.063 | — | — | — |
| Non-stimulated control | | 0.097 | 0.095 | 0.096 | — | — | — |
| Stimulated control | | 0.260 | 0.252 | 0.254 | 100 | 100 | 100 |
| CBD | 0.01% | 0.258 | 0.255 | 0.254 | 98.77 | 101.91 | 100.00 |
| | 0.025% | 0.248 | 0.246 | 0.249 | 92.64 | 96.18 | 96.84 |
| | 0.05% | 0.235 | 0.233 | 0.234 | 84.66 | 87.90 | 87.34 |
| | 0.1% | 0.227 | 0.222 | 0.226 | 79.75 | 80.89 | 82.28 |
| | 0.25% | 0.174 | 0.172 | 0.178 | 47.24 | 49.04 | 51.90 |
| | 0.5% | 0.09 | 0.092 | 0.091 | 0 | 0 | 0 |
| | 1% | 0.085 | 0.089 | 0.088 | 0 | 0 | 0 |
| | 2.5% | 0.084 | 0.081 | 0.087 | 0 | 0 | 0 |

| | | | |
|---|---|---|---|
| | Concentrations | Optical Densities observed for | Cell viability (% as compared |
| Samples | (weight %) | the 3 sets of experiments | to the negative control) |

| Blank | | 0.07 | 0.07 | 0.071 | — | — | — |
|---|---|---|---|---|---|---|---|
| Non-stimulated control | | 0.075 | 0.071 | 0.075 | — | — | — |
| Stimulated control | | 0.730 | 0.726 | 0.721 | 100 | 100 | 100 |
| CBD | 0.01% | 0.745 | 0.729 | 0.733 | 102.29 | 100.46 | 101.86 |
| | 0.025% | 0.756 | 0.731 | 0.728 | 103.92 | 100.76 | 101.08 |
| | 0.05% | 0.705 | 0.701 | 0.723 | 96.20 | 96.18 | 100.31 |
| | 0.1% | 0.732 | 0.702 | 0.716 | 100.24 | 96.34 | 99.23 |
| | 0.25% | 0.655 | 0.648 | 0.651 | 88.53 | 88.09 | 89.16 |
| | 0.5% | 0.120 | 0.119 | 0.123 | 6.95 | 7.33 | 7.43 |
| | 1% | 0.109 | 0.106 | 0.107 | 5.19 | 5.34 | 4.95 |
| | 2.5% | 0.102 | 0.102 | 0.104 | 4.12 | 4.73 | 4.49 |

TABLE 12
Anti-inflammatory activity and cell viability
for mixture of NeutraZen 0.05% and cannabidiol

| | Concentrations | Optical Densities observed for | | | NO production (% as compared | | |
|---|---|---|---|---|---|---|---|
| Samples | (weight %) | the 3 sets of experiments | | | to the negative control) | | |
| Blank | | 0.061 | 0.065 | 0.063 | — | — | — |
| Non-stimulated control | | 0.097 | 0.095 | 0.096 | — | — | — |
| Stimulated control | | 0.260 | 0.252 | 0.254 | 100 | 100 | 100 |
| CBD | 0.01% | 0.26 | 0.258 | 0.259 | 100.00 | 103.82 | 103.16 |
| | 0.025% | 0.25 | 0.252 | 0.251 | 93.87 | 100.00 | 98.10 |
| | 0.05% | 0.234 | 0.231 | 0.235 | 84.05 | 86.62 | 87.97 |
| | 0.1% | 0.227 | 0.22 | 0.228 | 79.75 | 79.62 | 83.54 |
| | 0.25% | 0.174 | 0.178 | 0.171 | 47.24 | 52.87 | 47.47 |
| | 0.5% | 0.09 | 0.102 | 0.098 | 0 | 4.46 | 1.27 |
| | 1% | 0.089 | 0.095 | 0.092 | 0 | 0 | 0 |
| | 2.5% | 0.088 | 0.087 | 0.086 | 0 | 0 | 0 |

| | | | |
|---|---|---|---|
| | Concentrations | Optical Densities observed for | Cell viability (% as compared |
| Samples | (weight %) | the 3 sets of experiments | to the negative control) |

| Blank | | 0.07 | 0.07 | 0.071 | — | — | — |
|---|---|---|---|---|---|---|---|
| Non-stimulated control | | 0.075 | 0.071 | 0.075 | — | — | — |
| Stimulated control | | 0.730 | 0.726 | 0.721 | 100 | 100 | 100 |
| CBD | 0.01% | 0.717 | 0.738 | 0.731 | 98.02 | 101.83 | 101.55 |
| | 0.025% | 0.724 | 0.734 | 0.726 | 99.08 | 101.22 | 100.77 |
| | 0.05% | 0.737 | 0.73 | 0.725 | 101.04 | 100.61 | 100.62 |
| | 0.1% | 0.733 | 0.726 | 0.721 | 100.46 | 100.00 | 100.00 |
| | 0.25% | 0.721 | 0.72 | 0.723 | 98.56 | 99.08 | 100.31 |
| | 0.5% | 0.126 | 0.131 | 0.124 | 7.76 | 9.16 | 7.59 |
| | 1% | 0.126 | 0.125 | 0.123 | 7.74 | 8.24 | 7.43 |
| | 2.5% | 0.128 | 0.122 | 0.12 | 8.14 | 7.79 | 6.97 |

TABLE 13
Anti-inflammatory activity and cell viability
for mixture of NeutraZen 0.1% and cannabidiol.

| | Concentrations | Optical Densities observed for | | | NO production (% as compared | | |
|---|---|---|---|---|---|---|---|
| Samples | (weight %) | the 3 sets of experiments | | | to the negative control) | | |
| Blank | | 0.061 | 0.065 | 0.063 | — | — | — |
| Non-stimulated control | | 0.097 | 0.095 | 0.096 | — | — | — |
| Stimulated control | | 0.260 | 0.252 | 0.254 | 100 | 100 | 100 |
| CBD | 0.01% | 0.262 | 0.258 | 0.251 | 101.23 | 103.82 | 98.10 |
| | 0.025% | 0.265 | 0.254 | 0.255 | 103.07 | 101.27 | 100.63 |
| | 0.05% | 0.227 | 0.228 | 0.226 | 79.75 | 84.71 | 82.28 |
| | 0.1% | 0.215 | 0.212 | 0.214 | 72.39 | 74.52 | 74.68 |
| | 0.25% | 0.18 | 0.164 | 0.175 | 50.92 | 43.95 | 50.00 |
| | 0.5% | 0.091 | 0.097 | 0.092 | 0 | 1.27 | 0 |
| | 1% | 0.088 | 0.09 | 0.091 | 0 | 0 | 0 |
| | 2.5% | 0.09 | 0.089 | 0.088 | 0 | 0 | 0 |

| | | | |
|---|---|---|---|
| | Concentrations | Optical Densities observed for | Cell viability (% as compared |
| Samples | (weight %) | the 3 sets of experiments | to the negative control) |

| Blank | | 0.07 | 0.07 | 0.071 | — | — | — |
|---|---|---|---|---|---|---|---|
| Non-stimulated control | | 0.075 | 0.071 | 0.075 | — | — | — |
| Stimulated control | | 0.730 | 0.726 | 0.721 | 100 | 100 | 100 |
| CBD | 0.01% | 0.736 | 0.726 | 0.724 | 100.96 | 100.00 | 100.46 |
| | 0.025% | 0.728 | 0.722 | 0.722 | 99.68 | 99.39 | 100.15 |
| | 0.05% | 0.749 | 0.723 | 0.721 | 102.89 | 99.54 | 100.00 |
| | 0.1% | 0.711 | 0.715 | 0.718 | 97.10 | 98.32 | 99.54 |
| | 0.25% | 0.702 | 0.709 | 0.712 | 95.73 | 97.40 | 98.61 |
| | 0.5% | 0.131 | 0.133 | 0.128 | 8.56 | 9.47 | 8.20 |
| | 1% | 0.131 | 0.126 | 0.124 | 8.58 | 8.40 | 7.59 |
| | 2.5% | 0.133 | 0.121 | 0.116 | 8.93 | 7.63 | 6.35 |

Table 14 reports the calculated values of the IC50 of NO release, the IC50 of cell viability and the anti-inflammatory ratio for dexamethasone, cannabidiol, Neutrazen and mixtures combining cannbidiol/Neutrazen.

The results are obtained by a non-linear regression analysis of the curves obtained in FIGS. 1 to 6 of the different series of experiments.

TABLE 14
Calculated values of the IC50 of NO release, the IC50 of cellular
viability and the anti-inflammatory ratio for dexamethasone, cannabidiol,
Neutrazen and mixtures associating cannabidiol/Neutrazen.

| | IC50-NO | IC50-cell | Anti-inflammatory |
|---|---|---|---|
| Samples | release | viability | ratio |
| Dexamethasone | 2.98 ± 0.18 μM | 192.65 ± 4.25 μM | 64.65 |
| CBD | 0.319 ± 0.006% | 0.101 ± 0.005% | <1 |
| Neutrazen | >2.5% | >2.5% | — |
| CBD/Neutrazen (0.01% weight) | 0.272 ± 0.005% | 0.328 ± 0.003% | 1.20 |
| CBD/Neutrazen( 0.025% weight) | 0.234 ± 0.004% | 0.343 ± 0.005% | 1.46 |
| CBD/Neutrazen (0.05% weight) | 0.233 ± 0.005% | 0.372 ± 0.003% | 1.59 |
| CBD/Neutrazen (0.1% weight) | 0.218 ± 0.005% | 0.434 ± 0.003% | 1.99 |

The calculated results of the anti-inflammatory ratio in Table 14 show that the presence of Neutrazen, namely palmitoyl tripeptide-8, induces an improvement in the anti-inflammatory ratio of cannabidiol. Indeed cannabidiol has an anti-inflammatory ratio of less than 1, but associated with Neutrazen, the ratio is improved. A ratio of 1.99 is notably observed when cannabidiol is combined with a 0.1% weight Neutrazen solution.

FIG. 7 reports the isobolograms for inflammatory activity and cell viability.

The results obtained with the different mixtures of CBD and Neutrazen™ made it possible to calculate the pairs of concentrations corresponding to a 50% reduction in the production of NO and a 50% reduction in cell viability. These data have been transferred to FIG. 7, which represents the isobolograms corresponding to the experiment.

In the isobologram in FIG. 7a concerning the anti-inflammatory activity, the experimental curve obtained with the cannabidiol/Neutrazen™ concentration pairs representing a 50% decrease in NO production are below the observed theoretical line for additive effects (shown in dotted line).

In parallel with the isobologram in FIG. 7b concerning cell viability, the experimental curve obtained with the cannabidiol/Neutrazen™ concentration pairs representing a 50% decrease in cell viability is greater than the theoretical line of additive effects shown in dotted line.

These results demonstrate that the association of cannabidiol and Neutrazen™ (containing Palmitoyl hexapeptide-8) in a mixture involves synergistic anti-inflammatory activity and a concomitant decrease in cytotoxicity, resulting in an improved anti-inflammatory ratio.

### REFERENCES

1—Okoko T I. Oruambo IF Inhibitory activity of quercetin and its metabolite on lipopolysaccharide-induced activation of macrophage U937 cells. Food Chem Toxicol. 2009 April;47(4):809-12. doi: 10.1016/j.fct.2009.01.013.

2—Yazihan N I. Karakurt O. Ataoglu H. Erythropoietin reduces lipopolysaccharide-induced cell Damage and midkine secretion in U937 human histiocytic lymphoma cells. Adv Ther. 2008 May;25(5):502-14. doi: 10.1007/s12325-008-0055-5.

1-13. (canceled)

14. Association comprising or consisting of a mixture of cannabidiol or cannabigerol and palmitoyl tripeptide-8.

15. Association according to claim 14 comprising a mixture of:
cannabidiol or cannabigerol,
palmitoyl tripeptide-8,
butylene glycol,
dextran,
and water.

16. Association according to claim 14, comprising or consisting of a mixture of cannabidiol and palmitoyl tripeptide-8.

17. Association according to claim 14 comprising a mixture of:
cannabidiol,
palmitoyl tripeptide-8,
butylene glycol,
dextran,
and water.

18. Association according to claim 14 comprising or consisting of a mixture of cannabigerol and palmitoyl tripeptide-8.

19. Association according to claim 14 comprising a mixture of:
cannabigerol,
palmitoyl tripeptide-8,
butylene glycol,
dextran,
and water.

20. Association according to claims 14, in which:
cannabidiol or cannabigerol is present in an amount of 0.01% to 5%, preferably 0.25% to 1% in percentage by weight,
palmitoyl tripeptide-8 is present in an amount of 2 ppm to 20 ppm, preferably 4 ppm to 10 ppm by weight.

21. Association according to claim 14, additionally comprising acetyl hexapeptide-1

22. Association according to claim 14 comprising a mixture of:
cannabidiol or cannabigerol,
palmitoyl tripeptide-8,
butylene glycol,
dextran,
and water.
additionally comprising acetyl hexapeptide-1

23. Association according to claim 14, comprising at least one oil containing α-linolenic acids,
or one oil containing α-linolenic acids from Perilla Oil, Linseed Oil, Camelina Oil, Inca Inchi Oil, Chia Oil and Rosehip Oil from Chile.

24. Association according to claim 14 comprising a mixture of:
cannabidiol or cannabigerol,
palmitoyl tripeptide-8,
butylene glycol,
dextran,
and water.
and at least one oil containing α-linolenic acids, or one oil containing α-linolenic acids from Perilla Oil, Linseed Oil, Camelina Oil, Inca Inchi Oil, Chia Oil and Rosehip Oil from Chile

25. Association according to claim 14, with an α-linolenic acid content of 33% to 66% relative to the total fatty acids of the oil and with an omega-3/omega-6 ratio of 0.20 to 1.0.

26. Association according to claim 14 comprising a mixture of:
cannabidiol or cannabigerol,
palmitoyl tripeptide-8,
butylene glycol,
dextran,
and water.
with an α-linolenic acid content of 33% to 66% relative to the total fatty acids of the oil and with an omega-3/omega-6 ratio of 0.20 to 1.0.

27. Association according to claim 14, comprising a natural extract of Tasmannia Lanceolata.

28. Association according to claim 14 comprising a mixture of:
cannabidiol or cannabigerol,
palmitoyl tripeptide-8,
butylene glycol,
dextran,
and water
and comprising a natural extract of Tasmannia Lanceolata

29. Cosmetic composition, comprising the association according to claim 14, in a cosmetically acceptable medium, said composition optionally comprising at least one other ingredient, chosen in particular from: moisturizing agents, chemical filters, sun filters, in particular UV A filters, inorganic mineral sun filters, in particular titanium dioxide, thermal waters, in particular Treignac® water.

30. Cosmetic composition, comprising the association according to claim 14 comprising a mixture of:
cannabidiol or cannabigerol,
palmitoyl tripeptide-8,
butylene glycol,
dextran,
and water
in a cosmetically acceptable medium, said composition optionally comprising at least one other ingredient, chosen in particular from: moisturizing agents, chemical filters, sun filters, in particular UV A filters, inorganic mineral sun filters, in particular titanium dioxide, thermal waters, in particular Treignac® water.

31. Cosmetic composition, comprising the association according to claim 14, in a cosmetically acceptable medium, said composition optionally comprising at least one other ingredient, chosen in particular from: moisturizing agents, chemical filters, sun filters, in particular UV A filters, inorganic mineral sun filters, in particular titanium dioxide, thermal waters, in particular Treignac® waterCosmetic, said cosmetic composition being formulated for topical application, or topical application to the skin, or in the form of an emulsion, a cream, a gel, a dispersion, a serum, a mousse, a body milk or an anhydrous balm, a day cream, a night cream or an after-sun product.

32. Cosmetic composition, comprising the association according to claim 14 comprising a mixture of:
cannabidiol or cannabigerol,
palmitoyl tripeptide-8,
butylene glycol,
dextran,
and water
in a cosmetically acceptable medium, said composition optionally comprising at least one other ingredient, chosen in particular from: moisturizing agents, chemical filters, sun filters, in particular UV A filters, inorganic mineral sun filters, in particular titanium dioxide, thermal waters, in particular Treignac® water, said cosmetic composition being formulated for topical application, or topical application to the skin, or in the form of an emulsion, a cream, a gel, a dispersion, a serum, a mousse, a body milk or an anhydrous balm, a day cream, a night cream or an after-sun product.

33. Method for the cosmetic treatment of sensitive skin, comprising the topical application to healthy skin of an association according to one of claims 14 to reduce the sensations of stinging, tingling, itching or pruritus, burning, overheating, discomfort or tightness of the skin.
